(19) United States
(12) Patent Application Publication
(10) Pub. No.: US 2024/0309471 A1
(43) Pub. Date: Sep. 19, 2024

(54) SYSTEMS AND METHODS FOR DETECTING VIRAL INFECTIONS

(71) Applicant: QCDx LLC, Farmington, CT (US)

(72) Inventors: Spencer Ryan KEILICH, Farmington, CT (US); Triantafyllos TAFAS, Rocky Hill, CT (US)

(21) Appl. No.: 17/985,377

(22) Filed: Nov. 11, 2022

Related U.S. Application Data
(63) Continuation of application No. PCT/US21/32314, filed on May 13, 2021.
(60) Provisional application No. 63/135,905, filed on Jan. 11, 2021.
(60) Provisional application No. 63/024,679, filed on May 14, 2020.

(51) Int. Cl. C12Q 1/70; C12Q 1/6841

(52) U.S. Cl. CPC C12Q 1/701; C12Q 1/6841

(57) ABSTRACT

The present disclosure includes systems and methods for detection of viral infections. These systems and methods employ a fluorescence light sheet microscopy system that provides highly sensitive and specific detection and isolation of immune cells for early detection of viral infections.

### CROSS-REFERENCE

This application is a continuation of International Application No. PCT/US21/32314, filed May 13, 2021, which claims the benefit of U.S. patent application No. 63/024,679, filed on May 14, 2020, and U.S. patent application No. 63/135,905, filed on Jan. 11, 2021, each of which is incorporated herein by reference in its entirety.

### BACKGROUND

Viral infection of cells can be assessed by determining a presence of viral proteins or viral genes in the cells. For example, target cells can be stained with an antibody exhibiting specific binding to a viral protein to determine viral infection of the target cells. In another example, target cells can be lysed to extract nucleic acid samples from the target cells, and the nucleic acid samples can be analyzed, for example, via polymerase chain-reaction to determine viral infection of the target cells.

### INCORPORATION BY REFERENCE

Each patent, publication, and non-patent literature cited in the application is hereby incorporated by reference in its entirety as if each was incorporated by reference individually.

### BRIEF DESCRIPTION OF THE DRAWINGS

FIG. 1 is a schematic representation of the selective plane illumination microscopy (SPIM) imaging method of the present disclosure.

FIG. 2 shows immunofluorescence staining of SARS-COV-2 in Vero cells.

FIG. 3 shows immunofluorescence staining of SARS-COV-2 over time in mice infected with SARS-COV-2.

FIG. 4 shows the number of white blood cells positive for SARS-COV-2 over time.

FIG. 5 show the results of SARS-COV-2 detection using a method disclosed herein.

FIG. 6 shows immunofluorescent images of white blood cells (WBC) from human and mouse stained with antibodies against SARS-COV-2 infection.

### SUMMARY OF THE INVENTION

In some embodiments, the invention provides a method, comprising: (a) contacting a target cell with a heterologous detection moiety, wherein the heterologous detection moiety exhibits specific binding to a heterologous nucleic acid sequence in the target cell; and (b) directing a light sheet at a cross-section of the target cell to obtain a cross-sectional image of the target cell, wherein the light sheet comprises a wavelength sufficient to detect the heterologous detection moiety, wherein the heterologous detection moiety is complexed with the heterologous nucleic acid sequence in the target cell.

In some embodiments, the invention provides a system, comprising: a heterologous detection moiety exhibiting specific binding to a heterologous nucleic acid sequence in a target cell; and an imaging device comprising: (i) a sample holder configured to hold the target cell while the target cell is contacted by the heterologous detection moiety; and (ii) an optical source, wherein the optical source is configured to direct a light sheet at a cross-section of the target cell to obtain a cross-sectional image of the target cell, wherein the light sheet comprises a wavelength sufficient to detect the heterologous detection moiety, wherein the heterologous detection moiety is complexed to the heterologous nucleic acid sequence in the target cell, and wherein the optical source is operably connected to the sample holder and oriented to connect the imaging device to the sample holder.

### DETAILED DESCRIPTION

Current methods for detecting viral infection involve detecting analytes, such as viral nucleic acids (DNA and RNA), viral proteins, intact viral particles, and antibodies using a variety of methods including, for example, polymerase chain reaction (PCR), virus culture, enzyme-linked immunosorbent assay (ELISA), western blots, and serological antibody detection methods. Viral nucleic acid detection tests can be sensitive several days after onset of an infection, whereas antibody tests can require more than two weeks post-infection to be sensitive. Thus, alternative methods and systems for a rapid detection of viral infection in target cells can be helpful in the diagnosis of viral infection.

The (+) single-stranded (ss) RNA coronaviruses (CoV) can cause fatal human respiratory diseases, such as Severe Acute Respiratory Syndrome (SARS)-causing CoV and Middle East Respiratory Syndrome (MERS)-CoV. At the end of 2019, a new SARS strain, SARS-COV-2 (COVID-19), which can be about 86% identical to SARS-COV-1 at the amino residue level, emerged in humans. When a human SARS-COV gains entry through the respiratory tract, one or more cells, such as, airway epithelial cells, alveolar epithelial cells, vascular endothelial cells, immune cells, or alveolar macrophages, can be a target of the human SARS-COV and cause replication of the human SARS-CoV. For example, the immune cells can be proinflammatory monocyte-derived macrophages in the bronchoalveolar lavage fluid. Such SARS-COV-2 permissive cells can contribute to one or more conditions in a subject, for example, lung inflammation and viral dissemination to other organs.

Nucleic acid amplification tests via reverse transcription polymerase chain reaction (RT-PCR) in respiratory specimens can be used to diagnose SARS-COV-2 infection in a subject. The sensitivity of these tests can be low, and can be affected by the limit of detection, viral inoculum, timing of testing, or the sample collection site. In some cases, RT-PCR-based detection methods may not detect a presence of viral infection in one or more days immediately after exposure to a virus.

COVID-19 detection can rely on a variety of viral swab-based nucleic acid tests or blood-based antibody tests. Viral swabs are quick and non-invasive, but are not sensitive enough and do not provide information about how the patient's immune system is responding to virus. Antibody detection can determine whether a patient is building immunity to viral infection. COVID-19 diagnosis can be determined based on SARS-COV-2 RNA detection by RT-PCR in nasopharynx samples. Lower respiratory samples can have better viral RNA yield than upper respiratory samples. SARS-COV-2 RNA can also be detected in stool and blood, where the RNA can be a marker of severe illness.

The sensitivity of a single nasopharyngeal swab early in the course of disease can only be around, for example, 70%. Delayed diagnoses can lead to nosocomial transmissions. Improved detection methods can simultaneously improve care for current patients, provide a safer working environment for clinicians, and help prevent the incursion of occult COVID-19 into hospitals.

Described herein is a highly sensitive microscopic method for detecting rare immune cells, infection-associated proteins, and nucleic acids in subject infected with a virus. The blood of subject disclosed herein can carry viral nucleic acids and express infection-associated proteins, which can be detected using a method disclosed herein. A method disclosed can be used, for example, for early screening of an asymptomatic subject; a subject that is at high-risk due to age, a pre-existing condition, or a comorbidity; a health care provider; a first line responder, a paramedic, police, fire fighters, and other individuals who have been exposed to a virus or have a viral infection.

### Systems and Methods for Detecting Heterologous Nucleic Acid Sequences in Cells

The methods of the present invention can comprise (a) contacting a target cell or a population of target cells with a heterologous detection moiety; and (b) directing a light to the target cell to obtain an image of the target cell (or the population of target cells). In some embodiments, the heterologous detection moiety exhibits specific binding to a heterologous nucleic acid sequence in the target cell or a population of target cells.

In some embodiments, the light is a light sheet for selective plane illumination microscopy (SPIM), for example, a Fluorescence Light Sheet Microscopy system (FLSM)). The light sheet can be directed to a cross-section of the target cell to obtain a cross-sectional image of the target cell. In some embodiments, the method can further comprise directing the light sheet at a plurality of cross-sections of the target cell, to obtain contiguous cross-sectional images of the target cell.

In some embodiments, the plurality of cross-sections can comprise at least or up to about 2 cross-sections, at least or up to about 5 cross-sections, at least or up to about 10 cross-sections, at least or up to about 15 cross-sections, at least or up to about 20 cross-sections, at least or up to about 25 cross-sections, at least or up to about 30 cross-sections, at least or up to about 40 cross-sections, at least or up to about 50 cross-sections, at least or up to about 60 cross-sections, at least or up to about 70 cross-sections, at least or up to about 80 cross-sections, at least or up to about 90 cross-sections, at least or up to about 100 cross-sections, at least or up to about 200 cross-sections, at least or up to about 300 cross-sections, at least or up to about 400 cross-sections, or at least or up to about 500 cross-sections.

In some embodiments, a spacing or distance between two adjacent cross-sections of the plurality of cross-sections can be at least or up to about 100 nanometers (nm), at least or up to about 200 nm, at least or up to about 300 nm, at least or up to about 400 nm, at least or up to about 500 nm, at least or up to about 1 micrometer (μm), at least or up to about 2 μm, at least or up to about 5 μm, at least or up to about 10 μm, at least or up to about 20 μm, at least or up to about 50 μm, at least or up to about 100 μm, at least or up to about 200 μm, at least or up to about 500 μm, or at least or up to about 1 mm.

In some embodiments, a method disclosed herein further comprises compiling the contiguous cross-sectional images to generate a composite image of the target cell. The composite image can be a three-dimensional (3D) image of the target cell. The composite image can be a video of the target cell. The composite image can be a visualized reconstruction of the target cell. In some embodiments, obtaining the image(s) (e.g., cross-sectional image(s)) can occur while the heterologous detection moiety and the target heterologous nucleic acid sequence are intact within the target cell without separately isolating the target heterologous nucleic acid sequence from the target cell.

In some embodiments, the heterologous detection moiety comprises a polynucleotide, wherein the polynucleotide comprises between about 5 to about 100 nucleobases. The polynucleotide of the heterologous detection moiety can comprise about 5 nucleobases to about 100 nucleobases. The polynucleotide of the heterologous detection moiety can comprise at least about 5 nucleobases. The polynucleotide of the heterologous detection moiety can comprise at most about 100 nucleobases. The polynucleotide of the heterologous detection moiety can comprise about 5 nucleobases to about 10 nucleobases, about 5 nucleobases to about 20 nucleobases, about 5 nucleobases to about 30 nucleobases, about 5 nucleobases to about 40 nucleobases, about 5 nucleobases to about 50 nucleobases, about 5 nucleobases to about 60 nucleobases, about 5 nucleobases to about 70 nucleobases, about 5 nucleobases to about 80 nucleobases, about 5 nucleobases to about 90 nucleobases, about 5 nucleobases to about 100 nucleobases, about 10 nucleobases to about 20 nucleobases, about 10 nucleobases to about 30 nucleobases, about 10 nucleobases to about 40 nucleobases, about 10 nucleobases to about 50 nucleobases, about 10 nucleobases to about 60 nucleobases, about 10 nucleobases to about 70 nucleobases, about 10 nucleobases to about 80 nucleobases, about 10 nucleobases to about 90 nucleobases, about 10 nucleobases to about 100 nucleobases, about 20 nucleobases to about 30 nucleobases, about 20 nucleobases to about 40 nucleobases, about 20 nucleobases to about 50 nucleobases, about 20 nucleobases to about 60 nucleobases, about 20 nucleobases to about 70 nucleobases, about 20 nucleobases to about 80 nucleobases, about 20 nucleobases to about 90 nucleobases, about 20 nucleobases to about 100 nucleobases, about 30 nucleobases to about 40 nucleobases, about 30 nucleobases to about 50 nucleobases, about 30 nucleobases to about 60 nucleobases, about 30 nucleobases to about 70 nucleobases, about 30 nucleobases to about 80 nucleobases, about 30 nucleobases to about 90 nucleobases, about 30 nucleobases to about 100 nucleobases, about 40 nucleobases to about 50 nucleobases, about 40 nucleobases to about 60 nucleobases, about 40 nucleobases to about 70 nucleobases, about 40 nucleobases to about 80 nucleobases, about 40 nucleobases to about 90 nucleobases, about 40 nucleobases to about 100 nucleobases, about 50 nucleobases to about 60 nucleobases, about 50 nucleobases to about 70 nucleobases, about 50 nucleobases to about 80 nucleobases, about 50 nucleobases to about 90 nucleobases, about 50 nucleobases to about 100 nucleobases, about 60 nucleobases to about 70 nucleobases, about 60 nucleobases to about 80 nucleobases, about 60 nucleobases to about 90 nucleobases, about 60 nucleobases to about 100 nucleobases, about 70 nucleobases to about 80 nucleobases, about 70 nucleobases to about 90 nucleobases, about 70 nucleobases to about 100 nucleobases, about 80 nucleobases to about 90 nucleobases, about 80 nucleobases to about 100 nucleobases, or about 90 nucleobases to about 100 nucleobases. The polynucleotide of the heterologous detection moiety can comprise about 5 nucleobases, about 10 nucleobases, about 20 nucleobases, about 30 nucleobases, about 40 nucleobases, about 50 nucleobases, about 60 nucleobases, about 70 nucleobases, about 80 nucleobases, about 90 nucleobases, or about 100 nucleobases. In some embodiments, the polynucleotide of the heterologous detection moiety comprises between about 15 to about 25 nucleobases. In some embodiments, the polynucleotide of the heterologous detection moiety comprises about 20 nucleobases.

In some embodiments, the heterologous detection moiety comprises a plurality of heterologous detection moieties. An individual heterologous detection moiety of the plurality of heterologous detection moieties can exhibit specific binding to a target heterologous nucleic acid sequence of the target cell. The individual heterologous detection moiety can be different from other target heterologous nucleic acid sequences of other heterologous detection moieties of the plurality of heterologous detection moieties. The plurality of heterologous detection moieties can comprise at least or up to about 2 different heterologous detection moieties, at least or up to about 3 different heterologous detection moieties, at least or up to about 4 different heterologous detection moieties, at least or up to about 5 different heterologous detection moieties, at least or up to about 10 different heterologous detection moieties, at least or up to about 15 different heterologous detection moieties, at least or up to about 20 different heterologous detection moieties, at least or up to about 25 different heterologous detection moieties, at least or up to about 30 different heterologous detection moieties, at least or up to about 35 different heterologous detection moieties, at least or up to about 40 different heterologous detection moieties, at least or up to about 45 different heterologous detection moieties, at least or up to about 50 different heterologous detection moieties, or at least or up to about 100 different heterologous detection moieties.

In some embodiments, a heterologous detection moiety provided herein can contain or be conjugated to a tag. Non-limiting examples of the tag can include a dye (e.g., tetramethylrhodamine isothiocyanate (TRITC), Quantum Dots, CY3 and CY5), a biotin-streptavidin conjugate, a magnetic bead, a fluorescent dye (e.g., fluorescein, Texas Red dye, rhodamine, or green fluorescent protein), a radiolabel (e.g., ³H, ¹²⁵I, ³⁵S, ¹⁴C, or ³²P), an enzyme (e.g., horse radish peroxidase or alkaline phosphatase), a calorimetric label such as colloidal gold, a colored glass bead, or a plastic bead (e.g., polystyrene, polypropylene, latex). The tag can be an optical tag (e.g., a fluorescent dye) that can be detected upon exposure to electromagnetic radiation (e.g., an optical light, such as a light sheet). The electromagnetic radiation can comprise one or more wavelengths from the electromagnetic spectrum including, but not limited to, x-rays (about 0.1 nanometers (nm) to about 10 nm; or about 10¹⁸ Hertz (Hz) to about 10¹⁶ Hz), ultraviolet (UV) rays (about 10 nm to about 380 nm; or about 8×10¹⁶ Hz to about 10¹⁵ Hz), visible light (about 380 nm to about 750 nm; or about 8×10¹⁴ Hz to about 4×10¹⁴ Hz), infrared (IR) light (about 750 nm to about 0.1 centimeters (cm); or about 4×10¹⁴ Hz to about 5×10¹¹ Hz), and microwaves (about 0.1 cm to about 100 cm; or about 10⁸ Hz to about 5×10¹¹ Hz).

In some embodiments, a heterologous detection moiety provided herein is delivered into the cell. The heterologous detection moiety can be delivered into the cell by, for example, transfection, transient transfection, stable transfection, or transduction. Non-limiting examples of delivery methods include, lipofection, electroporation, calcium phosphate precipitation, polyethyleneimine (PEI)-mediated transfection, DEAE-dextran mediated transfection, liposome-mediated transfection, particle gun technology, direct micro injection, use of cell permeable peptides, and nanoparticle-mediated delivery.

In some embodiments, a heterologous detection moiety as provided herein can comprise a deoxyribonucleic acid (DNA) sequence and/or a ribonucleic acid (RNA) sequence. The heterologous detection moiety can contain a synthetic nucleic acid sequence that is not expressed within or by the target cell. The heterologous detection moiety can comprise natural nucleobases. The heterologous detection moiety can comprise one or more analogues of a polynucleotide. Non-limiting examples of analogues can include 5-bromouracil, a peptide nucleic acid (PNA), a xeno nucleic acid, a morpholinos, a locked nucleic acid (LNA), a glycol nucleic acid, a threose nucleic acid, a dideoxynucleotide, cordycepin, 7-deaza-GTP, a CpG island, methyl-7-guanosine, a methylated nucleotide, inosine, thiouridine, pseudouridine, dihydrouridine, queuosine, and wyosine.

In some embodiments, the heterologous nucleic acid sequence is not a part of or is not derived from a genome of the target cell. In some embodiments, the heterologous nucleic acid sequence can be at least a portion of or can be derived from a heterologous organism, such as a bacteria or a virus. For example, the heterologous nucleic acid sequence can be a fragment of a viral genome, for example, a viral DNA sequence or a viral RNA sequence. Alternatively, the heterologous nucleic acid sequence can be derived from at least a portion of the viral genome, for example, the heterologous nucleic acid sequence can be a messenger RNA derived from at least a portion of the viral genome.

In some embodiments, the heterologous nucleic acid sequence can encode at least a portion of a viral gene and/or at least a portion of an intergenic region between the viral gene and an additional viral gene. In some embodiments, the heterologous nucleic acid sequence is native to the virus, e.g., not artificially introduced into the genome of the virus. For example, the heterologous nucleic acid sequence does not encode a non-viral protein (e.g., a human protein, a fluorescent protein, such as a green fluorescent protein (GFP), etc.). In some embodiments, the heterologous nucleic acid sequence can encode at least a portion of a viral protein.

Non-limiting examples of the viral gene that can be encoded by a heterologous nucleic acid sequence disclosed herein or can be detected by a heterologous detection moiety disclosed herein include orf1a, orf1ab, S (spike), 3a, 3b, E (envelope protein), M (matrix protein), p6, 7a, 7b, 8b, 9b, N (nucleocapsid), orf14, nsp1 (leader protein), nsp2, nsp3, nsp4, nsp5 (3C-like proteinase), nsp6, nsp7, nsp8, nsp9, nsp10 (growth-factor-like protein), nsp12 (RNA-dependent RNA polymerase, or RdRP), nsp13 (RNA 5′-triphosphatase), nsp14 (3′-to-5′ exonuclease), nsp15 (endoRNAse), and nsp16 (2′-O-ribose methyltransferase). In an example, the viral gene can encode a viral surface protein, such as S protein.

Non-limiting examples of the viral protein can include RNA-dependent RNA polymerase (RdRP), hemagglutinin (HA), nucleoprotein (N-protein), neuraminidase (NA), matrix protein (e.g., M1 or M2 proteins), NS1 protein, and nuclear export proteins, such as PB2, PB2, PB1-F2, PA, NP, M1, M2, NS1, and NEP/NS2.

### Viruses Disclosed Herein.

In some embodiments, a virus as disclosed herein can be a DNA virus or a RNA virus. The virus can be, for example, a double stranded DNA virus, a single stranded DNA virus, a double stranded RNA virus, a positive sense single stranded RNA virus, a negative sense single stranded RNA virus, a single stranded RNA-reverse transcribing virus (retrovirus), or a double stranded DNA reverse transcribing virus. Non-limiting examples of DNA viruses include cytomegalo virus (CMV), Herpex Simplex virus (HSV), Epstein-Barr virus, Simian virus 40, Bovine papillomavirus, Adeno-associated virus (AAV), Adenovirus, Vaccinia virus, and Baculo virus. Non-limiting examples of RNA viruses include, coronavirus, betacoronavirus, Semliki Forest virus, Sindbis virus, Poko virus, Rabies virus, Influenza virus, SV5, Respiratory Syncytial virus, Venezuela equine encephalitis virus, Kunjin virus, Sendai virus, Vesicular stomatitisvirus, and retroviruses.

Additional non-limiting examples of a virus include papovaviridae, adenoviridae, ampullaviridae, ascovirus, bicaudaviridae, clavaviridae, fuselloviridae, herpesviridae, herpesvirales, ascoviridae, ampullaviridae, asfarviridae, baculoviridae, fuselloviridae, globuloviridae, guttaviridae, hytrosaviridae, iridoviridae, lipothrixviridae, nimaviridae, poxviridae, tectiviridae, corticoviridae, sulfolobus, caudovirales, corticoviridae, tectiviridaea, ligamenvirales, globuloviridae, guttaviridae, turriviridae, baculovirus, hytrosaviridae, iridoviridae, polydnaviruses, mimiviridae, marseillevirus, megavirus, mavirus virophage, Sputnik virophage, nimaviridae, phycodnaviridae, pleolipoviruses, plasmaviridae, pandoraviridae, dinodnavirus, rhizidiovirus, salterprovirus, sphaerolipoviridae, anelloviridae, bidnaviridae, circoviridae, geminiviridae, genomoviridae, inoviridae, microviridae, nanoviridae, parvoviridae, spiraviridae, amalgaviridae, birnaviridae, chrysoviridae, cystoviridae, endornaviridae, hypoviridae, megabirnaviridae, partitiviridae, picobirnaviridae, quadriviridae, reoviridae, totiviridae, nidovirales, picornavirales, tymovirales, mononegavirales, bornaviridae, filoviridae, mymonaviridae, nyamiviridae, paramyxoviridae, pneumoviridae, rhabdoviridae, sunviridae, anphevirus, arlivirus, chengtivirus, crustavirus, wastrivirus, bunyavirales, feraviridae, fimoviridae, hantaviridae, jonviridae, nairoviridae, peribunyaviridae, phasmaviridae, phenuiviridae, tospoviridae, arenaviridae, ophioviridae, orthomyxoviridae, deltavirus, taastrup virus, alpharetrovirus, avian leukosis virus, rous sarcoma virus, betaretrovirus, mouse mammary tumor virus, gammaretrovirus, murine leukemia virus, feline leukemia virus, bovine leukemia virus, human T-lymphotropic virus, epsilon retrovirus, Walleye dermal sarcoma virus, lentivirus, human immunodeficiency virus 1 (HIV-1), simian and feline immunodeficiency viruses, spumavirus, simian foamy virus, orthoretrovirinae, spumaretrovirinae, metaviridae, pseudoviridae, retroviridae, hepadnaviridae, and caulimoviridae.

Non-limiting examples of coronavirus include alphacoronavirus, betacoronavirus, deltacoronavirus, and gammacoronavirus. Examples of alphacoronavirus include, for example, bat coronavirus CDPHE15, bat coronavirus HKU10, human coronavirus 229E, human coronavirus NL63, miniopterus bat coronavirus 1, miniopterus bat coronavirus HKU8, mink coronavirus 1, porcine epidemic diarrhea virus, rhinolophus bat coronavirus HKU2, and scotophilus bat coronavirus 512. Examples of betacoronavirus include, for example, betacoronavirus 1, hedgehog coronavirus 1, human coronavirus HKU1, Middle East respiratory syndrome-related coronavirus, murine coronavirus, pipistrellus bat coronavirus HKU5, rousettus bat coronavirus HKU9, severe acute respiratory syndrome-related coronavirus, tylonycteris bat coronavirus HKU4. Examples of deltacoronavirus include, for example, bulbul coronavirus HKU11, common moorhen coronavirus HKU21, coronavirus HKU15, munia coronavirus HKU13, night heron coronavirus HKU19, thrush coronavirus HKU12, white-eye coronavirus HKU16, wigeon coronavirus HKU20. Examples of gammacoronavirus include, for example, avian coronavirus, beluga whale coronavirus SW1. Additional examples of coronavirus can include MERS-COV, SARS-COV, and SARS-COV-2 (i.e., SARS-COV-2 or COVID-19).

Non-limiting examples of the influenza virus include influenza virus A, influenza virus B, influenza virus C, and influenza virus D. The influenza A virus can be of the subtype H1N1, H1N2, H2N2, or H3N2. The influenza B virus can be of the B/Yamagata/16/88-like lineage or the B/Victoria/2/87-like lineage.

### Target Cells Disclosed Herein.

In some embodiments, the target cell is derived from a subject. A subject can be a mammal. A subject can be a human. A subject can be a patient. A method disclosed herein can further comprise diagnosing a condition of the subject based on the cross-sectional image of the target cell. Non-limiting examples of the condition of the subject can include viral infection, bacterial infection, and parasitic infection.

In some embodiments, the target cell can be isolated from a biological sample of a subject. The biological sample can be, for example, a solid biopsy sample, a liquid biopsy sample, blood, urine, stool, plasma, menstrual fluid, saliva, lacrimal fluid, serum, cells, tissue, pleural fluid, synovial fluid, cerebrospinal fluid, DNA, RNA, hair, skin, or nails. For example, the biological sample is a blood sample of the subject, and the target cell can be an immune cell isolated from the blood sample. Non-limiting examples of the immune cell are a lymphoid cell, such as a B cell, a T cell, a cytotoxic T cell, a natural killer T cell, a regulatory T cell, a T helper cell, a natural killer cell, a cytokine induced killer (CIK) cell, a myeloid cell, a granulocyte, a basophil granulocyte, an eosinophil granulocyte, a neutrophil granulocyte, a monocyte, a macrophage, a red blood cell, a reticulocyte, a mast cell, a thrombocyte, a megakaryocyte, a dendritic cell, and a hematopoietic stem cell.

A method disclosed herein can provide increased or decreased sensitivity based on the type of target cell that is analyzed. For example, a method disclosed herein can exhibit increased or decreased sensitivity when the analyzed target cell is an immune cell. For example, a method disclosed herein can exhibit increased or decreased sensitivity when the analyzed target cell is a blood cell.

### Detection and Characterization of Immune Response to Viral Infection

Among immune cells, dendritic cells (DCs) are antigen-presenting cells (APCs) that are critical in the initiation of immune responses to control and/or eliminate viral infections. The first time a subject is exposed to a foreign antigen, for example, an antigen originating from a virus, the immune system can take up to two weeks to make an antibody blueprint and produce enough of the specific antibody to fight the infection. However, such an extended time period can be too late to know whether the subject is efficaciously responding to the viral infection. The process for creation of virus-specific antibodies begins with DCs responding nearly immediately to viral invasion. DCs can be isolated from peripheral blood as early as 1-4 days after initial infection and around the time human patients begin to show clinical symptoms of disease. Antibody secretion begins between days 7-9 post infection. After 15 days from the onset of infection 80% of all antibodies are compatible with the virus, at that point achieving maximum effectiveness for fighting the infection. High viral load in nasopharyngeal aspirate, with or without high viral load in serum, is a useful prognostic indicator of respiratory failure or death. Early treatment with an effective antiviral agent before day 10 can decrease the peak viral load, ameliorate symptoms, and improve outcome. Early treatment can also reduce viral shedding and thus the risk for transmission.

A method disclosed herein can detect a viral infection in a subject within about 1 day, about 2 days, about 3 days, about 4 days, about 5 days, about 6 days, about one week, about 8 days, about 9 days, about 10 days, about 11 days, about 12 days, about 13 days, about two weeks, about 2.5 weeks, about 3 weeks, about 3.5 weeks, or about one month after infection by, for example, a virus, a bacterium, or a parasite.

A method disclosed herein can detect, for example, a DC in a subject within about 1 day, about 2 days, about 3 days, about 4 days, about 5 days, about 6 days, about one week, about 8 days, about 9 days, about 10 days, about 11 days, about 12 days, about 13 days, about two weeks, about 2.5 weeks, about 3 weeks, about 3.5 weeks, or about one month post-infection. Such detection can be indicative of an infection in the subject.

### Systems, Devices, and Methods for Selective Plane Illumination Microscopy (SPIM)

Described herein is a system, device, and method for the detection of sub-populations of an analyte from a blood sample, e.g. rare immune cells, using a Fluorescence Light Sheet Microscopy system (FLSM). A system, device, or method disclosed herein use a selective plane illumination microscopy (SPIM) system to provide high sensitivity and specificity for the detection and isolation of individual analytes in, for example, the blood.

FLSM is a fluorescence microscopy technique in which a sample is illuminated by a laser light sheet. The laser light sheet is a laser beam that is focused in only one direction. In this case, the last light sheet is focused perpendicularly (i.e., orthogonally or 90 degrees to the direction of observation). The light sheet can be created using, for example, a cylindrical lens or by a circular beam scanned in one direction to create the light sheet. Using such a light sheet allows only the actually observed section of a sample to be illuminated. Therefore, this method can reduce the photodamage and stress induced on a living sample. Good optical sectioning capability can reduce the background signal and thus, create images with higher contrast, comparable to confocal microscopy. In addition, high depth penetration, low bleaching, and high acquisition speeds make light-sheet microscopy ideally suited for extended time-lapse experiments.

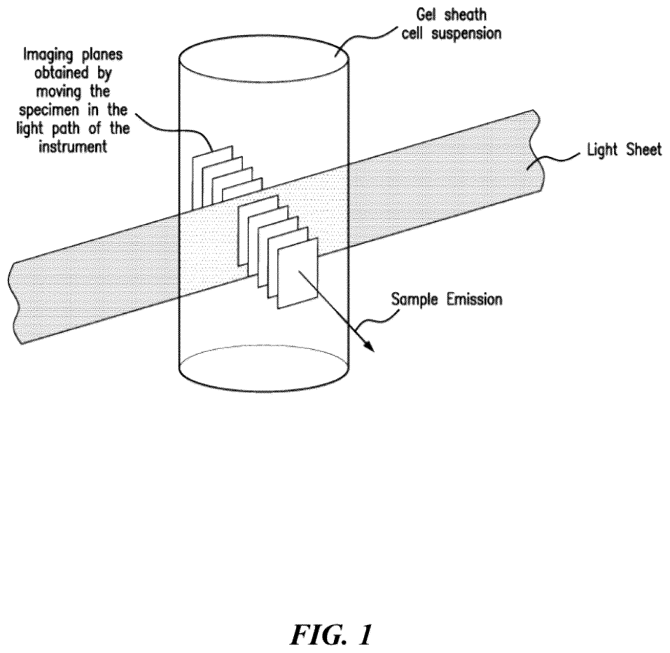

As shown in FIG. 1, a planar light sheet, such as a laser light sheet, is used to illuminate and penetrate a cell sample, such as the illustrated cylindrical gel sheath cell suspension and provides fluorescence excitation illumination. Multiple contiguous imaging planes can be obtained by moving the specimen in the light sheet path of the instrument. The light emission, such as the fluorescence emission, from the sample is observed perpendicular to the incident planar light sheets.

Using the technique depicted in FIG. 1, for example, a complete white blood cell (WBC) complement can be analyzed from a blood sample from a subject after fluorescent staining of cells. The cells can be analyzed in either a fixed or live state. The cell preparation can be made with cells morphologically intact, immobilized in a life-sustaining hydrogel such as agarose, and inside a transparent tubular fixture. The fixture can then be inserted in the FLSM optical path and visualization of the suspension can be performed by moving the fixture with a motorized, high-resolution stage. Cell images can then be collected and registered in 3D image stacks, separately for each fluorescent biomarker. Morphological information for different biomarkers can be digitized in 3D images at high-resolution and can deliver deep quantitation of protein expression after immunofluorescent (IF) staining or nucleic acids after fluorescence in situ hybridization (FISH) staining. The biomarker signals can then be quantitated, and the biomarkers can be used to phenotypically characterize the target cells.

A biomarker disclosed herein can be analyzed using, for example, immunofluorescence, western blot, qPCR, RT-PCR, or cell imaging. A biomarker analyzed herein can be, for example, a cell surface marker, an immune cell marker, a viral spike protein, any portion of a viral protein, or a nucleic acid.

In some embodiments, a method for characterizing and quantitating target cells in a blood sample comprises: (a) obtaining a blood sample from a subject; (b) preparing the sample by one or more of the following steps (i) through (vi), comprising: (i) centrifugation to separate cell layers from the blood serum layer (ii) removal of one of the cell layers; (iii) suspension of the removed layer from step (b)(ii) in a buffer; (iv) purification of the suspended layer of (b)(iii); (v) immobilization of the purified layer of (b)(iv), and (vi) immunostaining and/or fluorescence in situ hybridization (FISH) staining of the immobilized layer of (b)(v); (c) subjecting the prepared sample from (b) to selective plane image microscopy by scanning the sample with a light (for example a laser) sheet source at a multiple of cross sections to obtain contiguous cross-sectional images; (d) collecting a sufficient quantity of contiguous cross-sectional images; (e) compiling the contiguous cross-sectional images to produce a composite image; and (f) assessing the composite image to characterize and quantitate the blood sample for any target cells.

In some embodiments, a system for quantitating and characterizing target cells in a biological sample comprises: (a) a selective plane illumination microscope; (b) a sample fixture for containing the biological sample; (c) a detector for collecting the light image reflected orthogonally (90 degrees) to the illumination plane from the microscope; (d) a computer interface, and (e) a computer to compile the light images to create a 3-dimensional image.

The selective plane illumination microscope can comprise: (i) a laser light source, (ii) a light sheet generated from the laser light source, and (iii) an objective lens.

Non-limiting examples of analytes detectable by a system and device described herein include T-cells, DCs, white blood cells, B-cells, NK cells, or immune cells.

A biological sample from a subject can be analyzed to detect specific immune cells by using more than one immunofluorescence marker combined with single-molecule, RNA fluorescence in situ hybridization (smRNA FISH) that can target viral RNA. The smRNA FISH method can provide detection of, for example, rare DCs and other immune cells with viral infection, and can be used for the diagnosis of COVID-19 infections using SARS-COV-2 RNAs.

By utilizing smRNA FISH probes for different viruses, a system herein can be used for early detection of more than one viral species simultaneously infecting blood cells from a biological sample of a subject. The smRNA FISH method can be used for a subject who is suspected of viral exposure to different viral species that can cause respiratory disease, such as influenza or coronaviruses.

A method disclosed herein can be used for early screening of an asymptomatic subject who is suspected of exposure to, for example, a virus, bacterium, or parasite. Such an asymptomatic subject can include, for example, a high-risk subject with advanced age, pre-existing conditions, or a comorbidity for whom early detection of viral infection can be critical for disease management. These tests can be also used for early screening of health care providers and first responders including paramedics, police, and fire-fighting personnel.

An advantage of this method is the ability to detect target cells with high sensitivity early in the infection process when abundance of the target cells is very low, and provide results days before swab-based testing and more than a week earlier than antibody testing. The tests can have a 24-hour turnaround time after the specimen arrives at the facility.

In addition to early detection, a system, device, and method described herein can provide isolation of infected target cells for downstream, single-cell genomic and transcriptomic analysis with multiple applications in the study of viral diseases and the development of infectious disease treatments.

### EXAMPLES

### Example 1: Microscopy and RNA-FISH of Cells Infected with SARS-CoV-2

Vero cells (monkey kidney epithelial cells) are grown at 37° C. and 5% CO₂ in complete DMEM medium: Dulbecco's modified Eagle medium (DMEM) supplemented with 10% fetal bovine serum (FBS) and 1% penicillin-streptomycin (P/S). To ensure that the cell culture is mycoplasma free, cells are treated with MycoZap. SARS-COV-2 (NR-52281 SARS-related coronavirus 2, isolate USA-WA1/2020) is propagated in Vero-E6 cells and concentrated with a polyethylene glycol (PEG) to a titer of ˜1×10⁷ plaque forming units (PFU)/ml. Adeno 5 virus expressing the receptor for SARS-COV-2 (Ad5) and human Angiotensin Converting Enzyme 2 (ACE2) is used for infection.

8-10-week-old female C57BL/6J mice are inoculated with 2×10⁸ PFU of Ad5-hACE2 by intranasal instillation. Five days after Ad5 transduction, three mice are subsequentially infected intranasally with 2×10⁵ PFU of SARS-COV-2. Approximately 100 μL whole blood is collected before and 1, 3, 5, 10 days after SARS-COV-2 infection. In the Ad5-hACE2 mouse model, hACE2 was successfully expressed in lung tissue as shown by western blot. The presence of SARS-COV-2 is further validated using RT-PCR.

An RNA FISH probe was developed against the SARS-COV-2 Spike gene. The probe contains 48 individual oligomer-primers, as shown in TABLE 1.

TABLE 1

| SEQ ID No. | Sequence |
|---|---|
| 1 | gaattagtgtatgcaggggg |
| 2 | gggtaataaacaccacgtgt |
| 3 | ggtaagaacaagtcctgagt |
| 4 | gcatggaaccaagtaacatt |
| 5 | attggtcccagagacatgta |
| 6 | gggttatcaaacctcttagt |
| 7 | caccatcattaaatggtagg |
| 8 | tagacttctcagtggaagca |
| 9 | accaaaaatccagcctctta |
| 10 | ataagtagggactgggtctt |
| 11 | acttttgttgtttttgtggt |
| 12 | actctgaactcactttccat |
| 13 | gtgcaattattcgcactaga |
| 14 | ggtccataagaaaaggctga |
| 15 | tgaaattaccctgttttcct |
| 16 | ttaataggcgtgtgcttaga |
| 17 | aaaaccctgagggagatcac |
| 18 | ctaccaatggttctaaagcc |
| 19 | acctagtgatgttaatacct |
| 20 | agaagaatcaccaggagtca |
| 21 | ataataagctgcagcaccag |
| 22 | tcctaggttgaagataaccc |
| 23 | ctgtaatggttccattttca |
| 24 | gtcaagtgcacagtctacag |
| 25 | cgtacactttgtttctgaga |
| 26 | gattcctttttctacagtga |
| 27 | agattctgttggttggactc |
| 28 | ttcaccaaaagggcacaagt |
| 29 | tgcaaatctggtggcgttaa |
| 30 | cttcctgttccaagcataaa |
| 31 | cagcaacacagttgctgatt |
| 32 | ggagacactccataacactt |
| 33 | atttgtctgacttcatcacc |
| 34 | tcagcaatctttccagtttg |
| 35 | cgcagcctgtaaaatcatct |
| 36 | gattgttagaattccaagct |
| 37 | ttaccaccaaccttagaatc |
| 38 | ggtttgagattagacttcct |
| 39 | gcctgatagatttcagttga |
| 40 | accattacaaggtgtgctac |
| 41 | caccattagtgggttggaaa |
| 42 | tactactctgtatggttggt |
| 43 | ctggtgcatgtagaagttca |
| 44 | gtagactttttaggtccaca |
| 45 | gcctgttaaaccattgaagt |
| 46 | tagactcagtaagaacacct |
| 47 | gcaatgtctctgccaaattg |
| 48 | atcacggacagcatcagtag |

The full spike RNA target sequence is 1,673 nt in length and the chosen oligomers cover independent 20 nt oligomers. The 48 oligomers underwent basic local alignment search tool (BLAST) analysis to eliminate off-target hybridization. Each oligomer has a fluorescent tail of quasar 670. All white blood cells (WBCs) from each of 3 mice and each time-point blood samples were fluorescently immunostained in solution. Signals for 5 fluorescent markers were created by staining with antibodies against the common leukocyte antigen (rat-anti-mouse CD45) indirectly labeled with a goat-anti-rat secondary antibody labeled with Alexa Fluor 488; a macrophage cell anti-F4/80 marker directly conjugated with phycoerythrin (PE); a dendritic cell anti-CD11c marker (hamster-anti-mouse CD11c) indirectly labeled with goat-anti-hamster Alexa Fluor 594; and the RNA-fish spike probe fluorescently labeled with Quasar 670. Nuclei were counterstained by Hoechst 33342. After staining, the morphologically intact cells were immobilized in hydrogel into specimen fixtures that were loaded in the microscope (x, y, z, and rotational) microscope stage for 3-dimensional imaging. The fixture presents the cylindrical, transparent suspension of immobilized cells to the microscopy system optical path disclosed herein where the fixture was scanned in an automated fashion. Three-dimensional image stacks of the immobilized cells were acquired, individually for each of the 5 fluorescent markers. The 3D image stacks from each blood sample were analyzed and more than five hundred WBCs were counted to verify the presence of SARS-COV2 signals, totaling 1,500-2,000 cells from 3 mice and each one of the 5 fluorescent markers.

As described above, a cohort of 3 mice was tested longitudinally over a period of 10 days. A blood sample of 100 μL was collected retro-orbitally before SARS-COV-2 infection (0 DPI; days post-infection). Neither of the three blood samples had SARA-COV-2″ mRNA signatures. After the initial blood draw, the same three mice were infected with SARS-COV-2, and 100 μL blood was collected retro-orbitally, on 1, 3, 5, and 10 DPI.

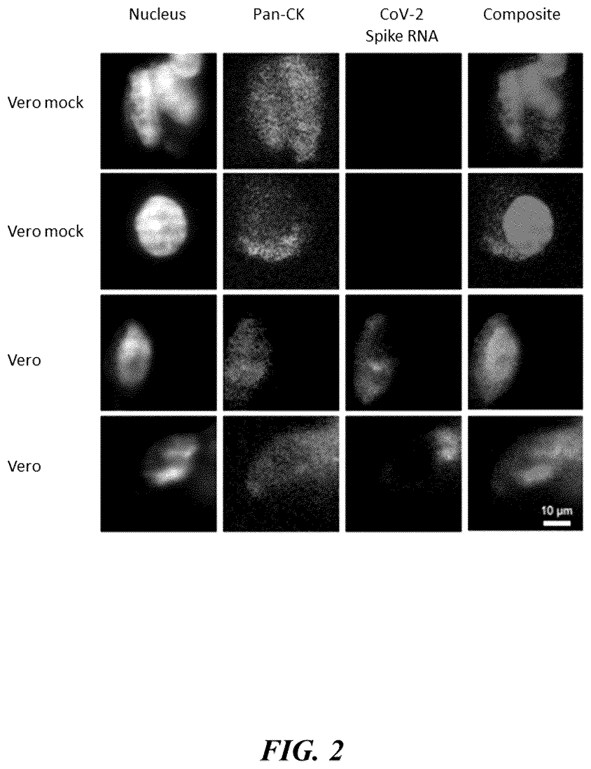

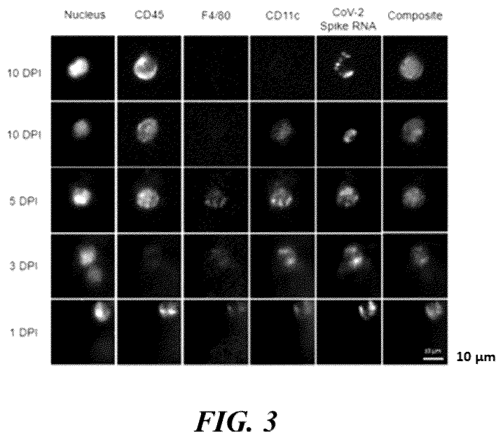

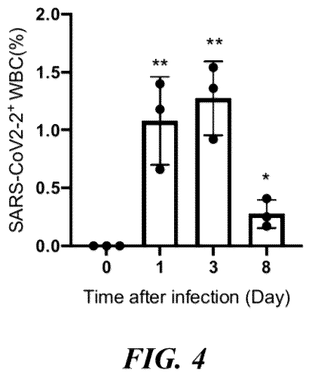

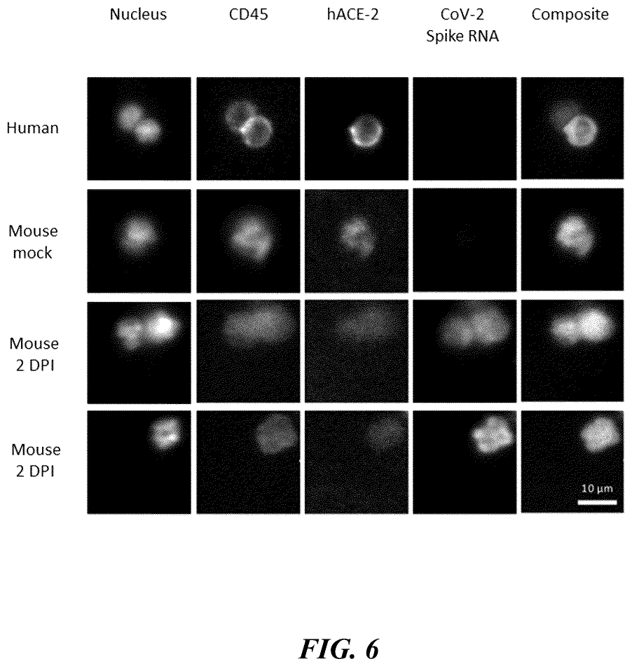

The results are shown in, for example, FIGS. 2-4. The SARS-COV-2-RNA probes were validated in heavily infected Vero cells as described in the present example (FIG. 2). CoV-2 Spike RNA was not detected in mock Vero cells that were not infected with SARS-COV-2-RNA (FIG. 2). A human blood sample without SARS-COV-2 infection was assessed as a control with high expression levels of hACE2. SARS-COV-2+/CD45+ double positive cells were detected in mice two days post infection (FIG. 6). Interestingly, hACE-2+/CD45+ double positive cells were observed in the blood of uninfected Ad5-hACE2 mice, but this observation was not present in all cells. (FIG. 6, row 2). For example, about 50% of the SARS-COV-2+/CD45+ double positive cells were also hACE2+, while the other 50% of the SARS-COV-2+/CD45+ double positive cells were hACE2-negative. CD45 leukocytes positive for SARS-COV-2 spike mRNA were observed since the first day post infection (1 DPI). SARS-COV-2⁺ cells increased in frequency to a peak of about 14% on 3 DPI and then declined gradually to about 6% on 10 DPI (FIG. 3 and FIG. 4). A statistically significant difference was observed between groups based on single factor Anova (f(4,10)=12.3, p=0.0007) comparing the change in mean frequency of spike positive cells overtime. The spike mRNA signal was robust with diverse morphology ranging from well-defined fish dots to disperse clouds of SARS-COV-2 signal positivity within in F4/80/CD11c double-positive and CD11c- or F4/80-positive cells (FIG. 3).

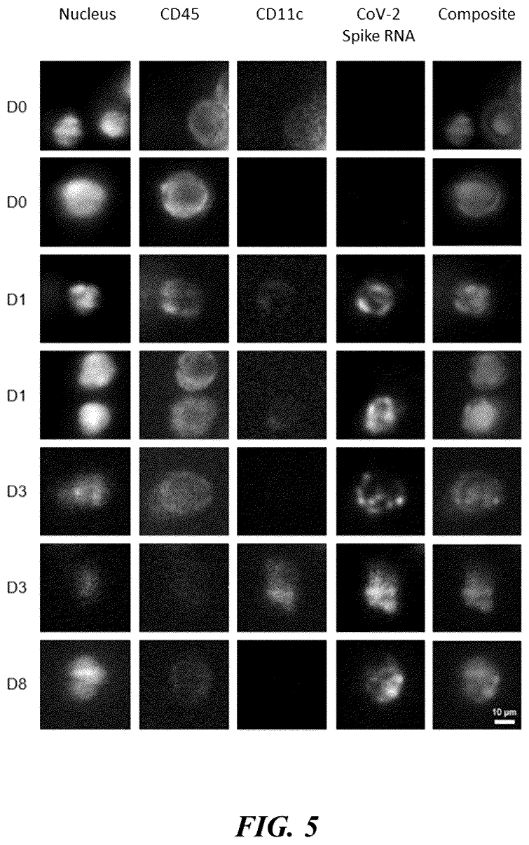

FIG. 5 shows additional images of samples assessed and summarized in FIG. 4. After validation of the mouse model and RNA-FISH probe, a cohort of 3 mice were tested longitudinally over a period of 8 days. Blood sample of 100 μl each mouse was collected retro-orbitally before SARS-COV-2 infection (0 DPI) and none of the three blood samples had SARS-COV-2+ Spike mRNA signatures in WBC (FIG. 4 and FIG. 5). The same three mice were infected with SARS-COV-2, and blood was collected on 1, 3, and 8 DPI. On the first day post infection (1 DPI or D1), leukocytes were positive for SARS-COV-2 Spike mRNA (1.08%), and SARS-COV-2++ cells increased slightly in frequency to a peak of about 1.27% on 3 DPI (D3) and then declined to about 0.28% on 8 DPI (D8) (TABLE 2, FIG. 4, and FIG. 5). The Spike mRNA signal was robust with diverse morphology ranging from well-defined FISH dots to disperse clouds of SARS-COV-2 signal positivity in CD45 and CD11c positive cells (FIG. 5). Another 50 μl of whole blood was collected at the same time in the same cohort for RNA extraction and quantitative RT-PCR (qPCR) detection of SARS-COV-2 RNA. No blood specimens tested positive for SARS-COV-2 by qPCR with a cut off threshold cycle (Ct) set at 40.

TABLE 2

| | | Spike | | | |
|---|---|---|---|---|---|
| | Sample ID | RNA+ | Nuclei | Ratio | Averages |
| Day 0 | M1 | 0 | 2571 | 0.00% | 0.00% |
| | M2 | 0 | 1000 | 0.00% | |
| | M3 | 0 | 1000 | 0.00% | |
| Day 1 | M1 | 32 | 2710 | 1.18% | 1.08% |
| | M2 | 22 | 3357 | 0.66% | |
| | M3 | 32 | 2292 | 1.40% | |
| Day 3 | M1 | 24 | 2606 | 0.92% | 1.27% |
| | M2 | 28 | 1822 | 1.54% | |
| | M3 | 56 | 4117 | 1.36% | |
| Day 8 | M1 | 15 | 6114 | 0.25% | 0.28% |
| | M2 | 10 | 2450 | 0.41% | |
| | M3 | 2 | 1183 | 0.17% | |

As demonstrated herein, RNA-FISH imaging was used to detect SARS-COV-2 RNA and dissemination kinetics in mouse blood circulation. SARS-COV-2 RNA-positive immune cells (e.g., leukocytes, such as CD11c cells) appeared as early as one day after infection and continued through day 10 post infection. The results presented herein show that SARS-COV-2-permissive leukocytes contributed to systemic viral dissemination, and RNA-FISH combined with Fluorescence Light Sheet Microscopy (FLSM) was able to detect SARS-COV-2 in blood specimens.

### Example 2: RNA Extraction and Quantitative Reverse-Transcription PCR

Total RNA is isolated from 50 μL of the whole blood per mouse and 20 mg of left lung using an RNAeasy mini-prep kit. Reverse transcription is performed using a Primescript™ RT reagent kit.

Quantitative PCR (qPCR) is performed with gene-specific primers and SYBR green.

The following PCR cycling program is used: 10 min at 95° C., and 40 cycles of 15 see at 95° C. and 1 min at 60° C.

Only day 1, blood specimens test positive for SARS-COV-2 by qPCR with a threshold cycle (CT) of 35-36, which is close to the limit of detection.

### Embodiments

The following non-limiting embodiments provide illustrative examples of the invention, but do not limit the scope of the invention.

Embodiment 1. A method, comprising: (a) contacting a target cell with a heterologous detection moiety, wherein the heterologous detection moiety exhibits specific binding to a heterologous nucleic acid sequence in the target cell; and (b) directing a light sheet at a cross-section of the target cell to obtain a cross-sectional image of the target cell, wherein the light sheet comprises a wavelength sufficient to detect the heterologous detection moiety, wherein the heterologous detection moiety is complexed with the heterologous nucleic acid sequence in the target cell.

Embodiment 2. The method of embodiment 1, further comprising directing the light sheet at a plurality of cross-sections of the target cell and thereby obtaining contiguous cross-sectional images of the target cell.

Embodiment 3. The method of embodiment 2, further comprising compiling the contiguous cross-sectional images to generate a composite image of the target cell.

Embodiment 4. The method of embodiment 3, wherein the composite image is a three-dimensional (3D) image of the target cell.

Embodiment 5. The method of any one of embodiments 1-4, wherein the light sheet comprises a laser light sheet.

Embodiment 6. The method of any one of embodiments 1-5, wherein the heterologous detection moiety comprises a polynucleotide, wherein the polynucleotide comprises from about 10 to about 30 nucleobases.

Embodiment 7. The method of any one of embodiments 1-6, wherein the polynucleotide comprises from about 15 to about 25 nucleobases.

Embodiment 8. The method of any one of embodiments 1-7 1, wherein the heterologous detection moiety comprises a plurality of heterologous detection moieties, wherein an individual heterologous detection moiety of the plurality of heterologous detection moieties exhibits specific binding to a target heterologous nucleic acid sequence that is different from other target heterologous nucleic acid sequences of other heterologous detection moieties of the plurality of heterologous detection moieties.

Embodiment 9. The method of embodiment 8, wherein the plurality of heterologous detection moieties comprises at least about 5 different heterologous detection moieties.

Embodiment 10. The method of embodiment 8 or 9, wherein the plurality of heterologous detection moieties comprises at least about 10 different heterologous detection moieties.

Embodiment 11. The method of any one of embodiments 8-10, wherein the plurality of heterologous detection moieties comprises at least about 20 different heterologous detection moieties.

Embodiment 12. The method of any one of embodiments 8-11, wherein the plurality of heterologous detection moieties comprises at least about 40 different heterologous detection moieties.

Embodiment 13. The method of any one of embodiments 1-12, wherein the heterologous detection moiety is a RNA-fluorescence in situ hybridization (FISH) probe.

Embodiment 14. The method of any one of embodiments 1-13, wherein the heterologous nucleic acid sequence is derived from a viral nucleic acid sequence.

Embodiment 15. The method of embodiment 14, wherein the heterologous nucleic acid sequence is a mRNA sequence derived from the viral nucleic acid sequence.

Embodiment 16. The method of any one of embodiments 1-15, wherein the heterologous nucleic acid sequence is a viral nucleic acid sequence.

Embodiment 17. The method of any one of embodiments 1-16, wherein the heterologous nucleic acid sequence encodes at least a portion of a viral protein.

Embodiment 18. The method of embodiment 17, wherein the viral protein is a viral surface protein.

Embodiment 19. The method of any one of embodiments 1-18, wherein the target cell is derived from a subject, and wherein the method further comprises diagnosing a condition of the subject based on the cross-sectional image of the target cell.

Embodiment 20. The method of any one of embodiments 1-19, further comprising determining a presence of the heterologous nucleic acid sequence in the target cell based on the cross-sectional image of the target cell.

Embodiment 21. The method of any one of embodiments 1-20, wherein the target cell is derived from a subject, and wherein the method further comprises diagnosing a condition of the subject based on the cross-sectional image of the target cell.

Embodiment 22. The method of any one of embodiments 1-21, wherein the target cell is isolated from a biological sample of a subject.

Embodiment 23. The method of embodiment 22, wherein the biological sample is a blood sample of the subject.

Embodiment 24. The method of any one of embodiments 1-23, further comprising directing an additional light sheet at an additional cross-section of the target cell to obtain an additional cross-sectional image of the target cell, wherein the additional light sheet comprises an additional wavelength sufficient to detect a marker of the target cell.

Embodiment 25. The method of embodiment 24, further comprising compiling the cross-sectional image and the additional cross-sectional image into a composite image.

Embodiment 26. The method of any one of embodiments 1-25, wherein the target cell is a mammalian cell.

Embodiment 27. The method of any one of embodiments 1-26, wherein the target cell is a human cell.

Embodiment 28. The method of any one of embodiments 1-27, wherein the target cell is an immune cell.

Embodiment 29. A system, comprising: a heterologous detection moiety exhibiting specific binding to a heterologous nucleic acid sequence in a target cell; and an imaging device comprising: (i) a sample holder configured to hold the target cell while the target cell is contacted by the heterologous detection moiety; and (ii) an optical source, wherein the optical source is configured to direct a light sheet at a cross-section of the target cell to obtain a cross-sectional image of the target cell, wherein the light sheet comprises a wavelength sufficient to detect the heterologous detection moiety, wherein the heterologous detection moiety is complexed to the heterologous nucleic acid sequence in the target cell, and wherein the optical source is operably connected to the sample holder and oriented to connect the imaging device to the sample holder.

Embodiment 100. A method, comprising: (a) contacting a target immune cell with a heterologous detection moiety, wherein the heterologous detection moiety exhibits specific binding to a heterologous nucleic acid sequence in the target immune cell, wherein the heterologous nucleic acid sequence encodes a coronavirus protein or an influenza virus protein; and (b) directing a light to the target immune cell to obtain an image of the target cell, wherein the light comprises a wavelength sufficient to detect the heterologous detection moiety, wherein the heterologous detection moiety is complexed with the heterologous nucleic acid sequence in the target immune cell.

Embodiment 101. The method of embodiment 100, wherein the light is a light sheet.

Embodiment 102. The method of embodiment 101, further comprising directing the light sheet at a plurality of cross-sections of the target immune cell, to obtain contiguous cross-sectional images of the target immune cell.

Embodiment 103. The method of embodiment 102, further comprising compiling the contiguous cross-sectional images to generate a composite image of the target immune cell.

Embodiment 104. The method of embodiment 103, wherein the composite image is a three-dimensional (3D) image of the target immune cell.

Embodiment 105. The method of any one of embodiments 100-104, wherein the heterologous detection moiety comprises a polynucleotide, wherein the polynucleotide comprises between about 10 to about 30 nucleobases.

Embodiment 106. The method of embodiment 105, wherein the polynucleotides comprise between about 15 to about 25 nucleobases.

Embodiment 107. The method of any one of embodiments 100-106, wherein the heterologous detection moiety comprises a plurality of heterologous detection moieties, wherein an individual heterologous detection moiety of the plurality of heterologous detection moieties exhibits specific binding to a target heterologous nucleic acid sequence that is different from other target heterologous nucleic acid sequences of other heterologous detection moieties of the plurality of heterologous detection moieties.

Embodiment 108. The method of embodiment 107, wherein the plurality of heterologous detection moieties comprises at least f5 different heterologous detection moieties.

Embodiment 109. The method of any one of embodiments 107-108, wherein the plurality of heterologous detection moieties comprises at least 10 different heterologous detection moieties.

Embodiment 110. The method of any one of embodiments 107-109, wherein the plurality of heterologous detection moieties comprises at least 20 different heterologous detection moieties.

Embodiment 111. The method of any one of embodiments 107-110, wherein the plurality of heterologous detection moieties comprises at least 40 different heterologous detection moieties.

Embodiment 112. The method of any one of embodiments 100-111, wherein the heterologous detection moiety is a RNA-fluorescence in situ hybridization (FISH) probe.

Embodiment 113. The method of any one of embodiments 100-112, wherein the heterologous nucleic acid sequence is derived from a viral nucleic acid sequence.

Embodiment 114. The method of any one of embodiments 100-113, wherein the coronavirus protein or the influenza protein is a viral surface protein.

Embodiment 115. The method of any one of embodiments 100-114, wherein the target immune cell is derived from a subject, and wherein the method further comprises diagnosing a condition of the subject based on the image of the target immune cell.

Embodiment 116. The method of any one of embodiments 100-115, further comprising determining a presence of the heterologous nucleic acid sequence in the target immune cell based on the image of the target immune cell.

Embodiment 117. The method of any one of embodiments 100-116, wherein the target cell is isolated from a biological sample of a subject.

Embodiment 118. The method of embodiment 117, wherein the biological sample is a blood sample of the subject.

Embodiment 119. The method of any one of embodiments 100-118, further comprising directing an additional light to the target immune cell to obtain an additional image of the target immune cell, wherein the additional light comprises an additional wavelength sufficient to detect a marker of the target immune cell.

Embodiment 120. The method of embodiment 119, further comprising compiling the image and the additional image into a composite image.

Embodiment 121. The method of any one of embodiments 100-120, wherein the target immune cell is a mammalian cell.

Embodiment 122. The method of any one of embodiments 100-121, wherein the target immune cell is a human cell.

1-29. (canceled)

30. A method, comprising:
(a) contacting a target cell with a heterologous detection moiety, wherein the heterologous detection moiety exhibits specific binding to a heterologous nucleic acid sequence in the target cell; and
(b) directing a light sheet at a cross-section of the target cell to obtain a cross-sectional image of the target cell, wherein the light sheet comprises a wavelength sufficient to detect the heterologous detection moiety, wherein the heterologous detection moiety is complexed with the heterologous nucleic acid sequence in the target cell.

31. The method of claim 30, further comprising directing the light sheet at a plurality of cross-sections of the target cell and thereby obtaining contiguous cross-sectional images of the target cell.

32. The method of claim 31, further comprising compiling the contiguous cross-sectional images to generate a composite image of the target cell.

33. The method of claim 32, wherein the composite image is a three-dimensional (3D) image of the target cell.

34. The method of claim 30, wherein the heterologous detection moiety is conjugated to a tag that can be detected upon exposure to electromagnetic radiation.

35. The method of claim 30, wherein the heterologous detection moiety comprises a polynucleotide, wherein the polynucleotide comprises from about 10 to about 30 nucleobases.

36. The method of claim 35, wherein the polynucleotide comprises from about 15 to about 25 nucleobases.

37. The method of claim 30, wherein the heterologous detection moiety comprises a plurality of heterologous detection moieties, wherein an individual heterologous detection moiety of the plurality of heterologous detection moieties exhibits specific binding to a target heterologous nucleic acid sequence that is different from other target heterologous nucleic acid sequences of other heterologous detection moieties of the plurality of heterologous detection moieties.

38. The method of claim 37, wherein the plurality of heterologous detection moieties comprises at least about 5 different heterologous detection moieties.

39. The method of claim 30, wherein the heterologous detection moiety is a RNA-fluorescence in situ hybridization (FISH) probe.

40. The method of claim 30, wherein the heterologous nucleic acid sequence is derived from a genome of a virus, wherein the heterologous nucleic acid sequence is native to the virus and is not artificially introduced into a genome of the virus.

41. The method of claim 30, wherein the heterologous nucleic acid sequence is a mRNA sequence derived from a viral nucleic acid sequence.

42. The method of claim 30, wherein the heterologous nucleic acid sequence encodes at least a portion of a viral protein.

43. The method of claim 42, wherein the viral protein is a viral surface protein.

44. The method of claim 30, wherein the target cell is derived from a subject, and wherein the method further comprises diagnosing a condition of the subject based on the cross-sectional image of the target cell.

45. The method of claim 30, further comprising determining a presence of the heterologous nucleic acid sequence in the target cell based on the cross-sectional image of the target cell.

46. The method of claim 30, further comprising directing an additional light sheet at an additional cross-section of the target cell to obtain an additional cross-sectional image of the target cell, wherein the additional light sheet comprises an additional wavelength sufficient to detect a marker of the target cell.

47. The method of claim 46, further comprising compiling the cross-sectional image and the additional cross-sectional image into a composite image.

48. The method of claim 30, wherein the target cell is a human cell.

49. The method of claim 30, wherein the target cell is an immune cell.
